(19) United States
(12) Patent Application Publication
(10) Pub. No.: US 2007/0248547 A1
(43) Pub. Date: Oct. 25, 2007

(54) CASCADE MACROMOLECULAR CONTRAST AGENTS FOR MEDICAL IMAGING

(75) Inventors: Robert C. Brasch, Mill Valley, CA (US); Yanjun Fu, San Francisco, CA (US); Danute E. Nitecki, Berkeley, CA (US)

Correspondence Address: MORGAN, LEWIS & BOCKIUS, LLP, ONE MARKET SPEAR STREET TOWER, SAN FRANCISCO, CA, 94105, US

(73) Assignee: THE REGENTS OF THE UNIVERSITY OF CALIFORNIA, Oakland, CA (US)

(21) Appl. No.: 11/690,645

(22) Filed: Mar. 23, 2007

Related U.S. Application Data
(60) Provisional application No. 60/785,260, filed on Mar. 23, 2006.

(51) Int. Cl. A61K 49/10; C08L 75/08

(52) U.S. Cl. 424/9.322; 525/438

(57) ABSTRACT

The present invention provides macromolecular contrast media for diagnostic imaging modalities.

### CROSS-REFERENCE TO RELATED APPIICATIONS

This application claims priority under 35 U.S.C. §119(e) to U.S. Provisional Patent Application No. 60/785,260 filed Mar. 23. 2006, which is incorporated herein by reference in its entirety for all purposes.

### FIELD OF THE INVENTION

The invention relates to novel cascade polymers conjugated with signal-generating molecules, as diagnostic imaging contrast agents, for X-ray imaging (including computed tomography, CT) and magnetic resonance imaging (MRI).

### BACKGROUND OF THE INVENTION

Magnetic resonance imaging (MRI), and X-ray imaging including computed tomography (CT) as well as radiography and fluoroscopy, are most widely used modalities in modern medical imaging. Both CT and MRI have the advantages of high spatial resolution and capability of multidimensional scanning. Compared to MRI, CT imaging has ionization radiation due to use of X-rays, but it has a stunningly high speed in image acquisition (e.g. as short as 50-100 milliseconds per slice) and an attractive ease in machine operation.

Intravenously administered image contrast enhancing drugs, “contrast media” or “contrast agents”, are used extensively for both MRI and CT, two of the most widely employed diagnostic imaging modalities in modern medicine. The demand for these contrast agents is based upon their well-recognized efficiency for improved diagnosis in many disease states. Today MRI contrast agents are used in more than 30% of clinical cases, while CT contrast agents are employed in most cases due to close attenuation values in most organs and tissues. Undoubtedly, CT and MRI contrast agents have occupied the majority of the whole contrast media market.

With few exceptions, all currently used CT and MRI contrast agents are less than 1000 Da in molecular weight (e.g. Gd-DTPA, Gd-DTPA-BMA, Gd-DOTA, diatrizoate, Iohexol, Iopamidol Iodixanol), and belong to the class of small molecular contrast media (SMCM). Their biodistribution, diffusing into the extracellular fluid space exclusive of the normal central nervous system has been called a “non-specific” distribution. Applications of these SMCM to highlight disruptions of the blood-brain barrier have been particularly valuable. However, contrast agents with a blood pool, intravascular distribution, are considered superior, in many respects, to SMCM. Unique advantages of blood pool contrast media, formulated as macromolecules, include prolonged angiographic effect, potential to quantify vascular characteristics such as blood volume, and potential to detect and measure disruption of vascular integrity outside the central nervous system. These potentials, angiography, quantitation of blood volume, and macromolecular permeability/leakiness, plus treatment response measurements, have particular appeal for cancer characterization. Additionally, compared with small molecules, less doses are required with macromolecular contrast agents for achieving the same efficacy.

A number of MMCM formulations for magnetic resonance imaging (MRI) have been designed and tested, establishing in pre-clinical investigations their unique diagnostic potential; but no MMCM has demonstrated all the desirable characteristics to be successfully advanced to governmental approval and clinical practice. The current status of MMCM development for MRI is reviewed in detail in “Macromolecular Contrast Agents for MR Mammography” (Daldrup H E et al. European Radiology, 2003, 13: 354). Briefly summarized, different obstacles or shortcomings have been encountered for each candidate with no currently described MMCM formulation meeting, all requirements. Some intermediately-sized agents [molecular weight <50 kilodaltons (kDa), e.g. Gadomer—17, P792] are small enough for prompt elimination by glomerular filtration but are too small to exploit and define the macromolecular hyper-permeability of cancer microvessels, a potential consistently observed with molecules larger than 70 kDa. Conversely, large MMCM represented by albumin-(Gd-DTPA)₃₀ (MW ˜92 kDa) or Gd-loaded dextran or dendrimers based on PAMAM starburst polymers are considered potentially well-suited for detecting microvascular hyper-permeability, but their further development were hampered by several severe drawbacks. Albumin-(Gd-DTPA) is too large and metabolically inert to be completely excreted, even 13% remained in the body after 36 days. In addition, albumin and other protein-based MMCM also are potentially immunogenic. Dextran-(Gd-DTPA/DOTA) had low Gd loading (˜5% in mole ratio), and also very polydispersed in size (polydispersity index, PDI, up to 2) hence leading to the incomplete clearance of the higher molecular weight fractions. Polylysine-(Gd-DTPA/DOTA) has been still associated with the problem of its size heterogeneity originated mainly from polylysine itself. PAMAM dendrimer-based Gd complexes had a much improved size homogeneity but still suffered from the unacceptable body clearance profile. Iron-oxide based MMCM are sufficiently large for cancer microvessel characterization and need not be eliminated from the body because the iron is utilizable for hemoglobin synthesis following metabolic breakdown. However, besides having substantially larger sizes (15-150 nm in diameter), particulate iron oxides MMCM induce a strong T2* effect which forces administration of only low doses to avoid T2* effects when used for quantitative T1-weighted applications and thus yield unimpressive tumor enhancement. This problem of weak T1-weighted enhancement with iron oxides is not only observed in experimental breast cancer models but also in humans. The first clinical trial of Ultrasmall Super-paramagnetic Iron Oxide (USPIO) for tumor characterization in breast cancer patients was reported most recently, using Clariscan™ (a USPIO with hydrodynamic diameter of 11.9 nm), that tumors are only poorly enhanced. (Daldrup H E et al, Radiology. 2003, 229: 885). Contrast agents that exist in vivo in an equilibrium between protein-bound macromolecular species and unbound small-molecular species (e.g. MS-325, B22956/1) are problematic because the kinetics and thus the permeabilities to the different species cannot be unraveled. Other formulations are hindered by macromolecular polydispersity (e.g. Gd-DTPA-polylysine). The novel MMCM in this patent were invented to overcome all these problems.

Despite the current lack of an ideal MRI MMCM for clinical development, the clinical potential and advantages afforded by MMCM for tumor characterization are well demonstrated in experimental animal studies. A recently published article, “MRI characterization of tumors and grading angiogenesis using macromolecular contrast media: status report” (Brasch R C et al, European Journal of Radiology, 2000, 34: 148) reviewed the recent development of this field. MMCM, as repeatedly demonstrated experimentally with prototype macromolecules such as albumin-(Gd-DTPA)₃₀, can exploit the well-recognized macromolecular hyper-permeability of cancer microvessels to differentiate benign from malignant tumors, to grade angiogenesis as correlated with histologic assays, to grade the biological aggressiveness and pathologic grade of cancer and to monitor cancer treatment responses, even within hours of treatment initiation. Four various human breast cancer and ovarian cancer models transplanted in rodents, this method has been used to specifically differentiate benign tumors from malignant ones, and to grade low or high malignancy of the latter, further to quantitatively evaluate tumor response to chemotherapy or anti-angiogenic treatments, which would be considerably significant in the early diagnosis and treatment of tumors in clinical practice. But these applications, demonstrated in animal models, cannot be achieved yet in patients due to the lack of a clinically suitable and governmentally approved MMCM.

In X-ray Imaging (mainly CT), there remains similar or even greater needs for macromolecular water-soluble contrast agents.

The typical clinical dose of small molecular iodinated CT contrast agents (˜5 mmol iodine/Kg body weight) is much more than that of gadolinium-based MRI contrast agents (0.1 mmol Gd/Kg body weight), thus requiring iodinated contrast agents to have more demanding tolerability and safety profiles in human subjects. However, the linear relationship between signal enhancement and contrast agent concentration within a very wide range of iodine concentrations is very attractive, especially in the quantitative studies based on imaging data.

Comparing various elements with high atomic number Z=39-82 comprehensively in the respects of opacification efficacy, biocompatibility and chemical modifiability, iodine atom (Z=53) remains now our primary choice as the radiopaque atom in our formulations of macromolecular CT contrast agents (Fu Y, Nitecki D E, Brasch R C, unpublished material).

In the field of CT MMCM, previous studies were limited and mainly involved the iodinated hydroxyethyl starch, carboxymethyl dextran derivative with triiodobenzoic acid, vinyl copolymers from acrylamide and hydrophilic triiodo monomers, and iodinated micelle, which were proven to be either physico-chemically undesirable, e.g. highly heterogeneous in molecular weight, poor in the content of radiopaque moiety, highly viscose, not stable; or to be poorly tolerated due to toxicity immunogenicity, or chronic accumulation in the body.

Desired water-soluble MMCM constructs for MRI and X-ray imaging, are expected to overcome all the deficiencies of existing formulations and incorporate all of following characteristics:
(a) sufficiently large molecular weight/size to yield a primary blood pool distribution and to allow definition of pathologic cancer microvessel leakiness,
(b) complete and timely bodily elimination,
(c) size monodispersity,
(d) biodegradability, when required for elimination,
(e) high dose-efficiency,
(f) biocompatibility and good tolerance,
(g) appropriate physico-chemical properties including good solubility, moderate osmolality and viscosity, heat-sterilizability, and storage stability.
(h) The constructs should also consist of readily-available, non-exotic, and relatively inexpensive components, preferentially components used previously as human pharmaceuticals and known to be well tolerated.

It is well known that polyethyleneglycol (PEG) has a number of unique properties. PEG binds water molecules via hydrogen-bonding, conferring an unusually large hydrodynamic size relative to molecular weight. PEG, buffered by the large quantities of water molecules bound to its surface, tends to exclude all other macromolecules and remains “unseen” by the body's immune system, thus has extremely low immunogenicity and antigenicity. Attachment of PEG to proteins, making them “stealth”, can dramatically prolong their blood half-life while substantially reducing immunogenicity. PEG is readily available in different sizes, inexpensive and nearly monodisperse, its polydispersity index (PDI) can be as low as 1.01 or even lower. In addition, PEG has good solubility in water and also in organic solvents such as methylene chloride, a highly useful characteristic in practical synthesis.

PEG has been incorporated into macromolecular drugs for human use as early as 1990; the PEG conjugate to adenosine deaminase (ADA), commercially known as ADAGEN, was first FDA-approved pegylated enzyme for intravenous use to treat ADA-deficient Severe Combined Immunodeficiency Syndrome in 1990. In this invention, we also chose PEG (or its analogs) as the backbone of our MMCM constructs.

But PEG has only two functional groups at both termini available for derivatization, thus appropriate amplifying strategy needs to be adopted to produce sufficient reactive termini (such as NH₂ groups). Cascade polymers (for example, cascade polyaminoacids as “amplifiers” with the varying generation and multiple terminal groups were introduced to two ends of PEG via various linkages (e.g. carbamate, amide, ester, disulfide, phosphates carbonate, etc). Subsequently signal-enhancing moieties (e.g. iodinated or Gd-based small contrast molecules) were attached to the terminal groups of “PEG amplifier”, yielding a new class of water-soluble macromolecular contrast agents.

Although components (PEG, cascade polymers, signal-enhancing groups and biodegradable linkages) we brought together in this invention have individually been exploited and evaluated previously, these same carefully-selected components have not been assembled in an as optimized and advantageous manner as we invented. In the literature about PEG-containing MMCM, PEG has been introduced, without exception, for the modification of either side chains of linear macromolecules (e.g. poly-L-lysine) or surface groups of cascade macromolecules (e.g. polyamidoamine cascade polymers) in an uncontrollable manner. The number of attached PEG can vary greatly.

In this invention, we introduced PEG into the center of MMCM as an initiation core of the cascading polymer, yielding genuinely well-defined structures and thus highly-reproducible preparations which is one pivotal factor in the development of clinically-useful macromolecular contrast agents. The constructs described in this invention can be clinically efficacious, well-tolerated in patients, and economically feasible.

The availability of MMCM for patients will allow for numerous valuable applications which previously have only been possible for experimental animals. In general, MMCM-enhanced imaging for cancer patients will allow for individual characterization of tumors; for example, one patient's breast cancer, biologically less aggressive, could be differentiated from the tumor of another patient. Benign tumors could be more specifically differentiated from malignant counterparts than is now possible by non-invasive imaging. The grades of malignant tumors could be defined by quantitative microvessel characterization, as shown in animal models of human breast and prostate cancers. Perhaps most importantly, the response of cancers to various treatments, for example, radiation therapy or anti-angiogenesis drug therapy could be monitored by MMCM-enhanced imaging assays of microvessel characteristics. Significant changes in MMCM permeability of breast cancer models in animals have been detected as early as 24 hours after treatment initiation. Taken together, these potential benefits offered by MMCM-enhanced imaging indicate a high level of significance for these invented MMCM.

### SUMMARY OF THE INVENTION

The constructs of this invention have the following general Formula (I):
(R³-z-S²-w)-R²-(y-S¹-x)-R¹-(x-S¹-y)-R²-(w-S²-z-R³) (I)
wherein R¹ is a macro core with two identical functional terminal groups. In an exemplary embodiment, R¹ is a member selected from polyalkylene glycol and derivatives thereof. In one embodiment, R¹ has a molecular weight or an average molecular weight of about 100 to about 10,000,000 dalton.

R² is a cascade polymer amplifier component and has the formula: R²═U—(NH-Z)n, wherein U is a reproduction unit of the starting generation, and Z is a repeating unit of the next generation U—(NH-Z)n. The number of generations range from 0 to 10. The integer n represents the multiplicity of the reproduction unit. In an exemplary embodiment, n is selected from 2 to 6.

R³ is a signal enhancing group for medical imaging applications, which is a member selected from a paramagnetic chelate (e.g., for MRI applications), a radiopaque organically-bound iodide (e.g., for CT applications) and an iodinated contrast agent (e.g., for X-ray imaging). In an exemplary embodiment, the number of signal enhancing groups used for imaging ranges between about 2 to about 2048.

S¹ is a spacer group linking the macro core and the cascade amplifier. In an exemplary embodiment, S¹ is a member selected from substituted or unsubstituted straight chain or branched chain C₁-C₁₂ alkyl and substituted or unsubstituted C₃-C₁₂ cycloalkyl S¹ optionally comprises one or more oxygen atom, carbonyl group and/or imino group wherein the imino group is optionally substituted by a carboxymethyl group. The C₁-C₁₂ alkyl group is optionally mono- or polysubstituted with hydroxy, carboxy, sulfono, phosphono and/or C₁-C₄ alkoxy groups.

S² is a spacer group linking the signal-enhancing group to the cascade amplifier. In an exemplary embodiment, S² is a member selected from substituted or unsubstituted straight chain or branched chain C₁-C₁₂ alkyl and substituted or unsubstituted C₃-C₁₂ cycloalkyl. S² optionally comprises one or more oxygen atom, carbonyl group and/or imino group wherein the imino group is optionally substituted by a carboxymethyl group. The C₁-C₁₂ alkyl group is optionally mono- or polysubstituted with hydroxy, carboxy, sulfono, phosphono and/or C₁-C₄ alkoxy groups. X is a linker moiety between the macro core and the spacer group S¹. Y is a linker moiety between the spacer group S and the cascade amplifier. W is a linker moiety between the cascade amplifier and the spacer group S². Z is a linker moiety between the spacer group S² and the signal enhancing group. In an exemplary embodiment, each x, y, w and z is a member independently selected from amide, carbamate, hydrazide, ureido, thioureido, azo, azido, ester, thioester, carbonate, phosphoester, disulfide and the like.

Further features and advantages of the invention will become apparent from the description which follows.

### DETAILED DESCRIPTION OF THE INVENTION AND THE PREFERRED EMBODIMENTS

Referring to Formula (I) above, pharmaceutical agents of the present invention are those in which R¹ stands for linear polymers which are water-soluble, non-toxic, non-antigenic, and have one chemically-modifiable group at each end of the main chain. Preferentially R¹ stands for polyalkylene glycols and analogs, and their derivatives, including polyethylene glycol (PEG), the most commonly used one in this class of linear polymers. Aside from the backbone, R¹ here also includes the linkages and spacer groups between the backbone and amplifier components. The preferred polymerization degree of these linear polymers ranges from about 2 to about 1000 (corresponding to PEG with a molecular weight of about 100 to about 44000 Daltons), with particularly preferred polymerization degree of about 20 to about 500 (corresponding to PEG with a molecular weight of about 1000 to about 20000 Daltons). The term “polymer” is used herein to include oligomers. In an exemplary embodiment, the polydispersity index (PDI) of water-soluble linear polymers of use in the invention is less than about 2, with PDI of from about 1.00 to about 1.20 preferred, with PDI of from about 1.00 to about 1.05 particularly preferred, and with PDI of below 1.01 most preferred.

In an exemplary embodiment, R² refers to an amplifier with a cascade polymer structure. Preferred generations for these cascade polymer components are between about 1 to about 10, with about 2 to about 8 generations particularly preferred, and with about 2 to about 6 generations most preferred. The number of terminal modifiable groups range from about 2 to about 1000, with terminal groups of about 4 to about 400 particularly preferred, and further with terminal groups of about 4 to about 100 most preferred.

In another exemplary embodiment, R³ is an iodinated contrast agent for X-ray imaging, or a complex of a ligand with a paramagnetic ion capable of enhancing the contrast in MRI. R¹ residues are covalently bound to the amplifier R² via versatile linkages and spacer groups.

The cascade macromolecular contrast agents having Formula (I) are described in detail in the following subsections. Exemplary compounds of the invention include PEG12000-Gen4-IOB₃₂ for CT, and PEG1200-Gen4-(Gd4-(Gd-DOTA monoamide )₃₂ for MRI, which are described in Bioconjugate Chemistry 2006, 17(4): 1043-1056), the disclosure of which is incorporated herein by reference in its entirety.

Water-Soluble Linear Polymer Backbone (R²)

In a preferred embodiment, R¹ is a member selected from polyalkylene glycol and analogs and derivatives thereof. In one embodiment, R¹ has a structure according to Formula (II):
(Y—R⁵—X)—R⁴—(X—R⁵—Y) (II)
wherein X is a first linker moiety between the linear core R⁴ and the spacer groups R⁵. In an exemplary embodiment, each X is a member independently selected from amide, carbamate, hydrazide, ureido, thioureido, azo, azido, ester, thioester, carbonate, phosphoester, disulfide and the like. Y is a second linker moiety between spacer groups R⁵ and the cascade polymer amplifiers. In an exemplary embodiment, each Y is a member independently selected from amide, carbamate, hydrazide, ureido, thioureido, azo, azido, ester, thioester, carbonate, phosphoester, disulfide and the like.

In Formula (II), R⁴ represents polyalkylene glycol and its analogs, an exemplary backbone group of Formula (II).

In an exemplary embodiment, R⁴ represents polyalkylene glycol, its monomeric unit has a general formula below:
(CR⁶R⁷—CR⁸R⁹—O) (III)
in which R⁶, R⁷, R⁸, R⁹ are H, alkyl groups (C₁-C₆), or substituted alkyl groups (C₁-C₆). Exemplary monomer units include the following:
(CH₂—CH₂—O) i.e. PEG,
(CHMe—CH₂—O) i.e. polypropylene glycol (PPG),
(CHEt-CH₂—O).

Polyalkylene glycols based on these monomers include not only their homopolymers, but also their copolymers. Exemplary linear polymers of use include the following:
(CH₂—CH₂—O)ₙ, (CHMe—CH₂—O)ₙ, (CH₂—CH₂—O)ₙ₁—(CHMe—CH₂—O)ₙ₂, (CH₂—CH₂—O)ₙ₁—(CH₂—CHMe—O)ₙ₂.
   in which n, n¹ and n² are independently selected integers greater than 1.

In other embodiments, R⁴ represents the analogs of polyalkylene glycols, their monomer units can have structures according to Formulae (IV) and (V):
(CR⁶R⁷—CR⁸R⁹—CR¹⁰R¹¹—O) (IV)
(CR⁶R⁷—CR⁸R⁹—CR¹⁰R¹¹—CR¹²R¹³—O) (V)

R⁶, R⁷, R⁸, R⁹, R¹⁰, R¹¹, R¹², R¹³ are independently selected H, alkyl groups (C₁-C₆), or substituted alkyl groups (C₁-C₆).

The polymer analogs based on these monomers (Formula IV and V) include not only their homopolymers, but also their copolymers with each other and with polymers based on formula II. Exemplary structures include the following:
(CH₂CH₂CH₂—O)ₙ, (CH₂CH₂CH₂CH₂—O)ₙ, (CH₂CH₂—O)ₙ₁—(CH₂CH₂CH₂—O)ₙ₂, (CH₂CH₂—O)ₙ₁-(CH₂CH₂CH₂CH₂—O)ₙ₂.

Alternative embodiments of R⁴ groups are those in which the PEG (or analogs) chain is interrupted by 1 or more hetero atoms (B, N, S, Si, Se etc), exemplary structures include the following:
(O—CH₂CH₂)ₙ₁-S—(CH₂CH₂—O)ₙ₂,
(O—CH₂CH₂)ₙ₁-OSi(CH₃)₂O—(CH₂CH₂—O)ₙ₂.

Further alternative embodiments of R⁴ groups are those in which the PEG (or analogs) chain is interrupted by 1 or more chemical bonds (X) without a spacer, exemplary embodiments include the following:
(O—CH₂CH₂)ₙ₁-X—(CH₂CH₂—O)ₙ₂,
{(O—CH₂CH₂)ₘ-X)}ₙ.

X=amide, carbamate, hydrazide, ureido, thioureido, azo, azido, ester, thioester, carbonate, phosphoester, disulfide etc

Typical examples include:
(O—CH₂CH₂)ₙ₁-SS—(CH₂CH₂—O)ₙ₂, a disulfide-containing PEG, {(O—CH₂CH₂)ₘ-O—C(═O)}ₙ, a copolymer from condensation of PEG and phosgene, in which n is an integer greater than 1.

Some other alternative embodiments of R⁴ groups are those in which the PEG (or analogs) chain is interrupted by 1 or ore chemical bond(s) and by 1 or more spacers the spacer may be an aliphatic or aromatic group (C₁-C₂₀), exemplary embodiments include the following:
(O—CH₂CH₂)ₙ₁-X—R¹⁴—X—(CH₂CH₂—O)ₙ₂.
{(O—CH₂CH₂)ₘ-X—R¹⁴—X)}ₙ,

X=amide, carbamate, hydrazide, ureido, thioureido, azo, azido, ester, thioester, carbonate, phosphoester, disulfide; R¹⁴=aliphatic or aromatic group (C₁C₂₀)

Typical examples include the following
(O—CH₂CH₂)ₙ-NHCOCH₂CH₂CONH—(CH₂CH₂—O)ₙ, and
   {(O═)C—(O—CH₂CH₂)ₘ-O—C(═O)-lysine}ₙ,
      a water-soluble poly(ether urethane) copolymer from the condensation of PEG bis(carbonylchloride) and L-lysine (Kohn J et al. Bioconjugate Chemistry, 1993, 4: 54)

Of note, in the condensation polymerization of PEG and another bifunctional monomer, a little excess PEG may be used so as to keep two hydroxyl group exposed at the termini of resulting copolymer.

All these linear polymers mentioned above are based on PEG itself or PEG analogs or their both derivatives. Among them, PEG distinguishes itself due to its unique properties: high water solubility, extremely low antigenicity and immunogenicity, FDA-approved utility as a component of various pharmaceuticals via intravenous or oral administration, unusually large exclusion volume in water, availability of high size-homogeneity (PDI as low as 1.01) solubility in various common organic solvents, etc. All these merits make PEG most desirable as the backbone component of our macromolecular contrast agents described in this invention.

In this invention, “PEG” refers to several basic types regarding its end groups (see below, “n” is the degree of polymerization)
PEG diol HO—(CH₂CH₂O)ₙ-H
PEG bisamine H₂N—(CH₂CH₂O)ₙ₋₁-CH₂CH₂NH₂
PEG diacid HOOCCH₂O—(CH₂CH₂O)ₙ₋₂-CH₂COOH
PEG dialdehyde OCHCH₂O—(CH₂CH₂O)ₙ₋₂-CH₂CHO
PEG dithiol HS—(CH₂CH₂O)ₙ₋₁-CH₂CH₂SH

(PEG here virtually means polyethylene glycol minus terminal functional groups.

In Formula II, X refers to the linkage between PEG (or its analogs) and the amplifer part. Generally, X can be classified into two categories non-biodegradable and biodegradable linkages.

For example, non-biodegradable linkages may be an amide, carbamate, hydrazide, ureido, thioureido, azo, azido, etc, biodegradable linkages may be an ester, thioester, carbonate, phosphoester, disulfide, short peptide sequence susceptible to enzymes, etc.

Of note, biodegradable linkages are necessary when the macromolecular contrast agents based on non-biodegradable linkages may not be able to be cleared adequately from the body. A number of biodegradable linkages with different degree of degradability, apparent to those skilled in the art, can be utilized in these cases. For example, to introduce an ester bond between R⁴ and R³, the esters with different neighboring groups (electron-withdrawing or electron-pushing groups) and thus different instability can be used:
—NH(CH₂)₅COO— <—NH(CH₂)₂COO— <—NHCH₂COO— <—OCH₂COO— (arranged by the instability order).

In Formula II, R⁵ stands for the spacer groups between the linkage X and the amplifier. The spacer group may be either a straight-chain or a branched-chain structure. Exemplary R⁵ groups are those which include a straight chain within their structures, either as the entire spacer group or as the backbone of a branched-chain group. The straight chain may be a chain of carbon atoms or of carbon atoms interrupted with one or more hetero atoms such as oxygen atoms, sulfur atoms or nitrogen atoms. Most common examples include alkyl diamine (C₂ to C₆), amino alcohol (C₂ to C₆), glycyl, β-alanyl, 6-aminohexanoic acid, cystamine etc.

Cascade Polymers as the Amplifier

In Formula I, R² represents generally a cascade polymer as the amplifier. Its reproduction units include, but not limited to, amino acids, amino alcohols, polyalcohols. polyamines, polyhydroxy carboxylic acids and so on. Exemplarily, R² represents nitrogen-containing cascade polymers as following (Formula II): 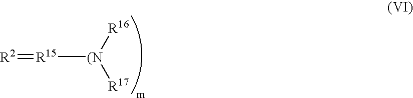
R¹⁵ (N—R¹⁶)ₘ Reproduction unit
R¹⁵ Reproduction unit (residue)
N Nitrogen atom
R¹⁶ H, alkyl (C₁-C₁₀), or acyl groups (C₂-C₁₀), each optionally containing 1-3 carboxy, or 1-3 oxygen atoms, or 1-5 hydroxy groups
m Multiplicity of the reproduction unit

Exemplary embodiments of the reproduction unit R¹⁵ (N—R¹⁶)ₘ are L-lysine, L-ornithine, other synthetic amino acids containing one carboxy group and ≦=2 amino groups.

One synthetic t-Boc amino acid derivative is given below (prepared from readily-available materials):

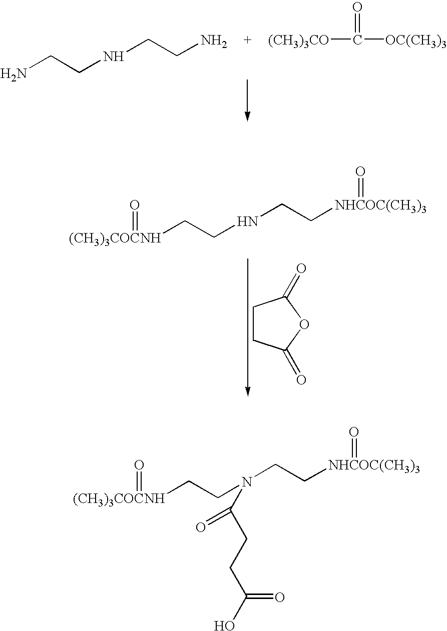

Compared to L-lysine, this synthetic amino acid has two advantages; no need to consider DL-isomers due to absence of chiral atoms, equivalent amino groups instead of different amino groups in lysine (α-NH₂ and ε-NH₂).

Exemplary R² group with Formula VI at least include (but is not limited to) cascading polyaminoacids, cascading polyamido amines, cascading polyalkyleneimines, which are examples of nitrogen-containing cascading polymers.

Alternative embodiments of R² group include cascading polyethers (Hall H et al, Journal of Organic Chemistry, 1987, 52: 5305), cascading polyesters (Frechet J M J et al, Bioconjugate Chemistry, 2002, 13: 453), cascading polyamido alcohols (Newkome G R et al, Journal of Organic Chemistry, 1985, 50: 2003), and so on.

Preferred generations for these cascade polymer components are between about 1 to about 10, with about 2 to about 8 generations particularly preferred, and with about 2 to about 6 generations most preferred. The number of terminal modifiable groups range from about 2 to about 1000, with terminal groups of about 4 to about 400 particularly preferred, and farther with terminal groups of about 4 to about 100 most preferred.

Signal-Enhancing Groups

R³ stands for a signal-enhancing group. In an exemplary embodiment, R³ has a structure according to Formula (VII):
X—R¹⁸—Y—R¹⁹ (VII)
wherein X is a linker moiety linking the spacer group (R¹⁸) and the amplifiers R² of Formula (I). Exemplary linker moieties X include amide, carbamate, hydrazide, ureido, thioureido, azo, azido, ester, thioester, carbonate, phosphoester, disulfide and the like. Y is a linker moiety linking the spacer group (R¹⁸) and the signal-enhancing group (R¹⁹). In an exemplary embodiments Y is a member selected from amide, carbamate, hydrazide, ureido, thioureido, azo, azido, ester, thioester, carbonate, phosphoester, disulfide and the like.

R¹⁸ is a spacer group between the signal-enhancing group (R¹⁹) and the amplifiers (R²). R¹⁹ is a signal-enhancing group.

i) CT Contrast Agents (“Triiodo” Species, Linker, Spacer)

Iodinated CT contrast agents are classified into two major categories, ionic agents and nonionic agents, which are characterized by inclusion of ionizable groups (i.e. carboxylate group) and neutral solubilization groups (i.e. short chain polyhydroxyalkyl or sugar groups), respectively. Nonionic contrast agents are more favored due to their substantially lower osmolality and better tolerability in patients, compared to their ionic counterparts.

The radiopaque moieties in our invention have exemplary formula VIII, IX, and X below, corresponding to 2,4,6-triiodo-5-acylamino-isophthalamides, 2,4,6-triiodo-3,5-acylamino-1-benzamides, 2,4,6-triiodo-1,3,5-benzamides (hereafter “triiodo”).

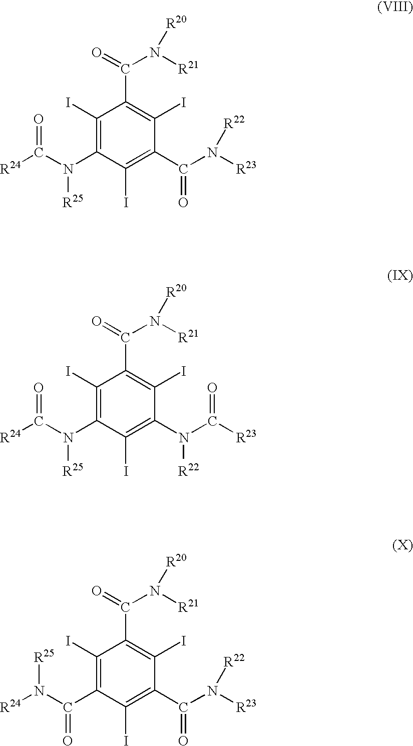

In Formulae (VIII), (IX) and (X), R²⁰, R²¹, R²², R²³, R²⁴, and R²⁵, independent of one another, stand for a hydrogen atom, or a carboxy group, or a straight chain or branched chain C₁-C₁₂ alkyl, or C₃-C₁₂ cycloalkyl, which is optionally interrupted one or more times by an oxygen atom, a carbonyl group and/or imino group wherein the latter is optionally substituted by a carboxymethyl group, and/or the C₁-C₁₂ alkyl is optionally mono- or polysubstituted by a hydroxy, carboxy, sulfono, phosphono and/or C₁-C₄ alkoxy group.

For each “triiodo” moiety, only one of these side chain groups (R²⁰, R²¹, R²², R²³, R²⁴, and R²⁵) is presented as the structure of a radical, which is the site where the “triiodo” moiety links to the spacer group (R¹⁸⁾.

Most of currently used nonionic CT contrast media are based on 2,4,6-triiodo-5-acylamino-isophthalamides structure with neutral polyhydroxyalkyl side chains, either monomeric or dimeric. Some of them containing 1,2-dihydroxy or 1,3-dihydroxy alkyl groups are given below as representative examples.

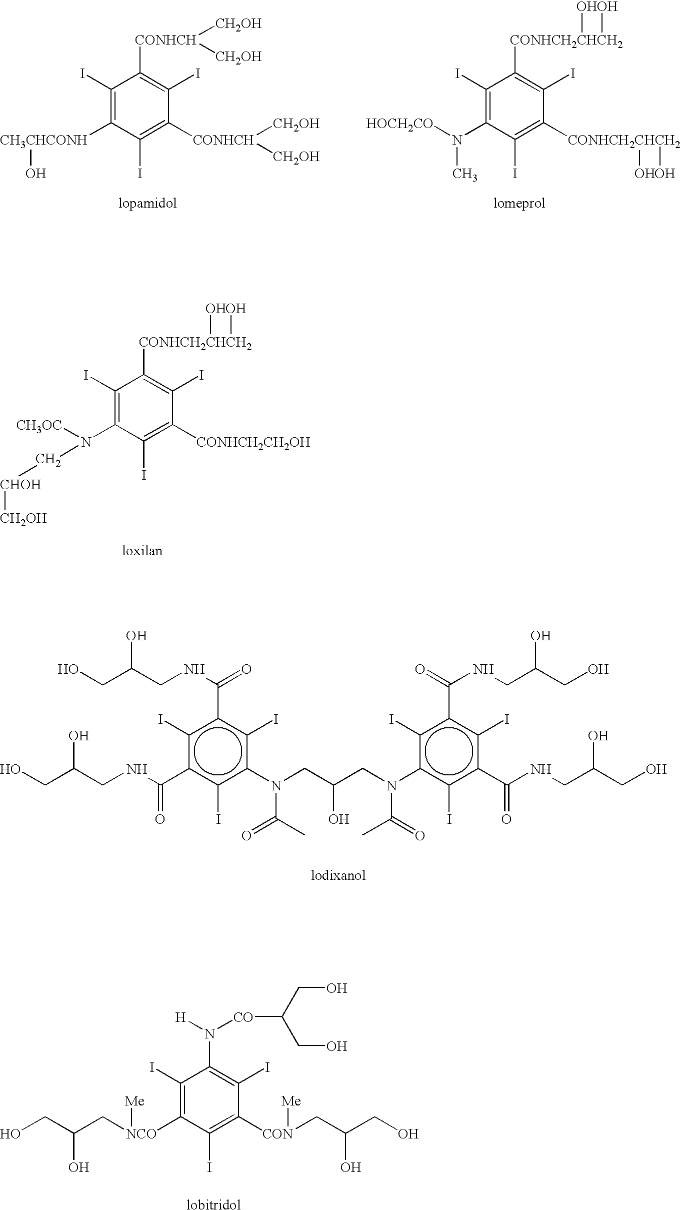

The five exemplary “triiodo” contrast media above can be derivatized after complete protection of all 1,2- or 1,3-dihydroxy alkyl groups by isopropylidene groups through ketalization. Our invented ketalization method of these iodinated contrast agents were proven highly yielding (generally >95%) and efficient (completed within 10-30 min). Subsequently, the remained hydroxyl or amido group can be selectively derivatized, allowing further attachment of these non-ionic iodinated moieties to terminal groups of the amplifiers.

Also, the appropriate triiodo moieties can be synthesized from very beginning, for example, starting from iodination of 5-amino isophthalamides, followed by chloration, amination and acylation, which is apparent to the skilled in the art.

Compared with non-ionic triiodo species, the ionic triiodo moieties are less preferred but still have their advantage like readily-available good solubility in water. They can be synthesized also by two approaches: one is the derivatization of existing small triiodo contrast agents, the other is the synthesis starting from simple materials.

Due to the much higher dose of CT contrast agents compared to their MRI counterparts, more demanding requirements of water solubility, tolerability etc are necessary for iodinated macromolecular contrast agents. The spacer group R18 in formula VII with hydrophilicity and non-immunogenicity are particularly preferred. Preferred R5 groups are those which include a straight chain within their structures, either as the entire spacer group or as the backbone of a branched-chain group. The straight chain may be a chain of carbon atoms or of carbon atoms interrupted with one or more hetero atoms such as oxygen atoms, sulfur atoms or nitrogen atoms. Exemplarily, aliphatic amino acids like glycine, β-alanine, or synthetic amino acids may be used as the spacer here. Two exemplary spacers can he synthesized by reacting monoester of tartaric acid (or glycolic anhydride) with 1-N-t-Boc ethylenediamine, shown as below.

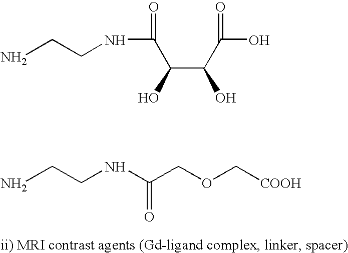

A wide variety of paramagnetic complexes may be used as group R³ in Formula I. Preferred complexes are chelates of a paramagnetic metal ion and a chelating agent. Chelates with high thermodynamic and kinetic stabilities are utilized since their ability to remain stable in vivo are necessary to meet the safety requirements as intravenously administrated MRI contrast agents. Macrocyclic chelating ligands are particularly preferred due to their high thermodynamic stability constants and low dissociation rate constants. The ligands must be bifunctional to permit both chelation with the paramagnetic metal ion and attachment to the spacer R¹⁸ of the construct, optionally through a linker group as appropriate. While a wide variety of ligands meets this description, a prominent example is 1,4,7,10-tetraazacyclododecane-N,N′, N″,N′″-tetraacetic acid (DOTA) and its analogs and derivatives. Further examples are 1,4,7,10-tetraazacyclododecane-N,N′,N″-triacetic acid (DO3A), diethylenetriaminepentaacetic acid (DTPA) and various analogs and derivatives of these ligands.

Open-chain MRI ligands (like DTPA) can be described by the following formula (XI): 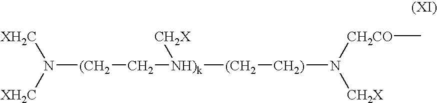
wherein X, being identical or different at each case, means the carboxy residue (COOH) or its derivatives (such as amide, ester etc), k=an integral number between 0-4.

Macrocyclic MRI ligands (Like DOTA) can be described by the following formula (XII): 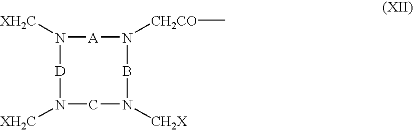
wherein X means the carboxy residue (COOH) or its derivatives: A, B, C, and D, being identical or different, mean the group (CH₂)ₐ-CH(R²⁵)—(CH₂)_{b}, wherein a and b are integers selected from 0, 1 and 2, with the proviso that the sum of a+b is greater than 1; R²⁵ means hydrogen, or a straight-chain, branched, saturated (or unsaturated) C1-C20 alkylene group.

Paramagnetic metal ions of a wide range are suitable for complexation with these ligands. Suitable metals are those having atomic numbers of 22-29 (inclusive), 42, 44 and 58-70 (inclusive). -Those having atomic numbers of 22-29 (inclusive) and 58-70 (inclusive) are preferred, and those having atomic numbers of 24-29 (inclusive) and 64-68 (inclusive) are more preferred. Examples of such metals are chromium (III) manganese (II), iron (II), iron (III), cobalt (II), nickel (II), copper (II), praseodymium (III), neodymium (III) samarium (III), gadolinium (III), terbium (III), dysprosium (III), holmium (III), erbium (III) and ytterbium (III). Chromium (III), manganese (II), iron (III) and gadolinium (III) are particularly preferred, with gadolinium (III) most preferred.

Since the first Gd-based contrast agent, Gd-DTPA (Magnevist) was introduced to clinical MRI examinations in early 1980's, numerous polyaminopolycarboxylic acids as chelating agents, either linear ones (like DTPA-BMA, BOPTA-DTPA), or cyclic ones (like DOTA, HP-DO3A) have been synthesized and characterized. A number of reviews (Desreux J F et al, Topics in Current Chemistry, 2002, 221: 123; Lauffer R B, Chemical Reviews, 1987, 87: 901) and many patents about the synthesis of new ligands based on these basic structures have been published, thus well known and apparent to those who skilled in the art.

Compared to carboxy derivatives of these ligands, the carbon-substituted derivatives are more stable after attachment to the macromolecules because, in the former cases, one carboxy group able to participate in the formation of Gd complex has to be sacrificed and used in the conjugation with the macromolecules, thus leading to the formation of less stable Gd complexes. Thus in our invented macromolecular constructs, the small carbon-substituted ligands are preferably chosen due to their higher chelating capability toward paramagnetic ions.

In the case of Gd-based contrast agents, the spacer groups (R¹⁸) between R¹⁹ and R² is widely optional. Generally. spacer groups with high hydrophilicity and non-antigenicity are preferred. For example, aromatic rings are less favored due to its potential immunogenicity.

The rigidness of spacer groups has a significant effect on the magnetic relaxivity of paramagnetic contrast agents. Generally, the more rigid the spacer group, the higher the per Gd relaxivity of the Gd-labelled macromolecules, which originates from the slowing tumbling rate of paramagnetic centers (small Gd-complex moieties) leading to the beneficially prolonged rotational correlation time of nearby water protons

The Synthetic Methods of Preferred Embodiments

To aid in describing synthetic methods of preferred embodiments, the synthetic routes of two macromolecular contrast agents, one for CT, one for MRI, can be found in FIG. 11 and FIG. 12 of U.S. Provisional Patent Application 60/785,260 filed Mar. 23, 2006.

As a typical exemple of R⁴ group (formula II), PEG diol may be reacted with 4-nitrophenyl chloroformate to give PEG biscarbonate, which then derivatized with mono-N-t-Boc alkyldiamine, followed by t-Boc deprotection, finally yielding PEG bisamine. An alternative of PEG bis(4-nitrophenyl carbonate) can be PEG his(N-succinimidyl carbonate). To assure the complete conversion of PEG diol hydroxyl groups to carbonate groups, 3-5 fold excess of chloroformate should be used. Biodegradable bonds such as disulfides maybe be incorported into PEG bisamine by reacting PEG biscarbonate with, for example, mono-N-t-Boc cystamine. Due to high reactivity of the amino group, the staring core molecule are most preferably PEG bisamines, compared with PEG diol, PEG dithiol, PEG diacid, PEG dialdehyde and so on. The quantification of amino groups may be conducted by classic 2,4,6-trinitrobenzenesulfonic acid (TNBS) method. PEG derivatives synthesis have been extensively reviewed in the literature, and well known for those who are skilled in the art. Here is a review book for example, Harris J M Ed., Poly(Ethylene Glycol) chemistry: biotechnical and biomedical applications, Plenum Press, New York, 1992.

Based on the PEG core above, cascade polymers can be synthesized. L-lysine is an example of the monomer. The cascade polylysine as a special type of polypeptide may be synthesized through classic t-Boc chemistry or Fmoc-chemistry. There are two reasons why we somewhat favor t-Boc chemistry here: a) Fmoc lysine derivatives are more expensive than their t-Boc counterparts; b) deprotection under basic conditions (e.g. piperidine) might not be suitable for the synthesis of some biodegradable constructs (e.g. ester-containing PEG12000-ester-Gen3 conjugate). Di-t-Boc lysine may be activated by several ways: direct DCC activation, symmetric anhydride, N-hydroxysuccinimidyl ester. 4-nitrophenyl ester, pentofluorophenyl ester etc. Symmetric anhydride may be most reactive, and the 4-nitrophenyl ester least reactive. The former method is material-consuming, and the latter may be time-consuming. The compromise is to adopt direct DCC activation method or N-hydroxysuccinimide ester method here. In the coupling reaction of the cascade amplifier with activated di-t-Boc L-lysine, the mole ratio of COOH/NH₂ is generally kept between 3-6, so as to assure there is no defected structure. Of note, a second repeated coupling needs to be conducted if any incompletion of coupling is detected. t-Boc deprotection can be carried out in the mixture of trifluoroacetic acid and methylene chloride (1:1 ratio often used) at room temperature within hours. In the literature, a similar compound PEG3400-amide-Gen4 was reportedly prepared via Fmoc chemistry in DMF (Choi J S et al, J. Am. Chem. Soc., 2000, 122: 474), which method is markedly different from ours.

The invented macromolecular constructs have a highly flexible design, since the size can be adjusted readily by modulating the length of PEG and the generations of cascade polymers. In general, the higher the generation, the more likely the existence of structure defects. Thus, most preferred number of generations for cascade amplifiers may be 2 to 5.

Small iodinated contrast agents, either ionic or nonionic but especially those non-ionic ones, may be derivatized via our invented method so as to avoid starting from the scratch and eliminate the corresponding long and tedious synthetic routes. The idea is to protect all 1,2- or 1,3-dihydroxy alkyl side chains of those suitable “triiodo” candidates via ketalization, then selectively modify the exposed and isolated one hydroxyl or amido group, producing synthetically-useful iodinated intermediates. After necessary activation, for example forming an N-hydroxysuccinimide ester group, these iodinated intermediates can be attached covalently to termini of the cascade amplifiers. Following deprotection of ketal groups in aqueous acid, macromolcular iodinated contrast agents can be obtained accordingly. The basics of iodinated contrast media chemistry are available in some recent reviews for example, Krause W et al, Topics in Current Chemistry, 2002, 222, 107; Sovak M. Radiocontrast agents. Spring-Verlag, Berlin, 1984), well-known for those skilled in the art.

Polyaminopolycarboxylic acid ligands with strong chelating ability, either linear or cyclic, are commonly used to complex Gd ion to produce MRI contrast agents. To maintain high chelating capability, carbon-substituted DTPA, DOTA derivatives can be used as the appropriate ligands for the synthesis of MRI contrast agents. To avoid the difficult coupling of highly charged ligands with the cascade amplifiers, all carboxy groups can be protected by t-butyl ester. After the coupling reaction, those protective groups can be removed by HCl (2 N) in suitable organic solvents. Gd chelation is then followed to produce final Gd-based MMCM. The basics of (Gd-based contrast media chemistry are available in some recent reviews (for example, Caravan P et al, Chemical Reviews, 1999, 99: 2293), well-known for those skilled in the art.

Chemical structure, synthesis, physical and biological properties of some exemplary cascading macromolecular contrast media in the invention can be found in FIG. 1 to FIG. 12 of U.S. Provisional Patent Application 60/785,260 filed Mar. 23, 2006.

Glossary
AES atomic emission spectrophotometry
Da Dalton
DO3A 1,4,7,10-tetraazacyclododecane-N,N′,N″-triacetic acid
DOTA 1,4,7,10-tetraazacyclododecane-N,N′,N″,N′″-tetraacetic acid
DTPA diethylenetriaminepentaacetic acid
DMF N,N-dimethylformamide
Et ethyl
ENU N-ethyl-N-nitrosourea
Fmoc fluorenylmethoxycarbonyl
fPv fractional plasma volume
Gen generation of the dendrimer
Gd gadolinium
HPLC high performance liquid chromatography
HP-SEC high performance size exclusion chromatography
IOB iobitridol (chemical name of a non-ionic contrast medium)
IOX Ioxilan (chemical name of a non-ionic contrast medium)
ICP inductive coupling plasma
IV intravenous
IVC inferior vena cava
KPS microvescular endothelial transfer coefficient
kDa kilodalton
MALDI matrix-assisted laser desorption ionization
Me methyl
MMCM macromolecular contrast media
MRI magnetic resonance imaging
MS mass spectrometry
MVD microvascular density
Mn number-average molecular weight
Mw weight-average molecular weight
NTA nitrilotriacetic acid
PDI polydispersity index
PEG polyethyleneglycol
p.i. post injection
RI refractive index
RI longitudinal relaxivity
SEC size exclusion chromatography
SMCM small molecular contrast media
TFA trifluoroacetic acid
TNBS trinitrobenzenesulfonic acid
T½ half-life
T1 longitudinal relaxation time
“triiodo” triiodinated aryl ring derivatives
t-Boc tert-butyloxycarbonyl
UV ultraviolet
CT computed tomography

The following examples are offered for purposes of illustration, and are intended neither to limit nor to define the invention in any manner.

### EXAMPLES

### Example 1

Preparation of PFEG6000-carbamate-Gen4-IOB conjugate

a) Bis(4-nitrophenyl Carbonate)-PEG6000 Preparation

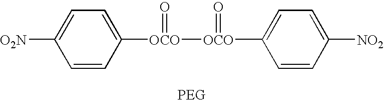

Dried PEG6000 (Mn 6470, 4.0 g, 0.62 mmol) was dissolved in 15 ml of anhydrous CH₂Cl₂ and 15 ml of dry pyridine then cooled to 0° C. To it was added 1.0 g of 4-nitrophenyl chloroformate (4.96 mmol) and 80 mg of 4-N,N-dimethylaminopyridine (DMAP). At 0° C. this mixture was stirred for 8 h. The reaction mixture was evaporated and precipitated by 80 ml of ether. Standing 0.5 h, the syrupy precipitate solidified, which was filtered and washed by ether. The crude product was dissolved in 1-2 ml of CH₂Cl₂ and precipitating it by 40 ml of anhydrous ether, this procedure was repeated twice. A white powder (4.06 g) was finally obtained.

Yield 97%. Elemental analysis (%):

| | C | H | N |
|---|---|---|---|
| Found | 54.03 | 8.52 | 0.50 |
| Theoretical | 54.34 | 8.82 | 0.41 |

b) α, ω-Bis(N-t-Boc-ethylcarbamoyl)-PEG6000

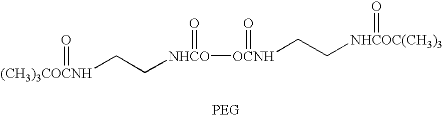

To a solution of 1-N-t-Boc-ethylenediamine (493 mg, 3.08 mmol) in 10 ml of CHCl₃ and 0.6 g of diisopropylethylamine (DIPEA, 4.64 mmol). 0.38 mmol of PEG6000 biscarbonate (2.58 g)) in 15 ml of CHCl₃ was added dropwise at 0° C. This reaction was allowed to continue 24 h at room temperature. The resulting mixture was evaporated and precipitated by 50 ml of ether, which was further purified by several dissolution-precipitation cycles using CHCl₃ and ether. A white powder (2.50 g) was obtained.

Yield 97%. Elemental analysis (%):

| | C | H | N |
|---|---|---|---|
| Found | 53.97 | 8.85 | 0.73 |
| Theoretical | 54.35 | 9.09 | 0.82 |

c) PEG6000-carbamate-Gen0.0 (i.e. PEG6000 bisamine)

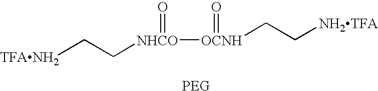

Boc-protected biscarbamate (3.02 g, 0.446 mmol) obtained above was dissolved in 8 ml of CH₂Cl₂ and cooled to 0° C., to which was added 8 ml of TFA. This mixture was stirred at 0° C. for 30 min and room temperature for another 2 h. Evaporation of the solution under 60° C. gave an oil which was solidified while standing at r.t. for several hours after adding 50 ml of anhydrous ether. The crude product was treated twice by dissolution-precipitation with CH₂Cl₂/ether, a white powder (2.93 g) was obtained.

Yield 98%. Elemental analysis (%):

| | C | H | N |
|---|---|---|---|
| Found | 52.76 | 8.60 | 0.71 |
| Theoretical | 53.08 | 8.85 | 0.82 |

d) PEG6000-carbamate-GEN4.0

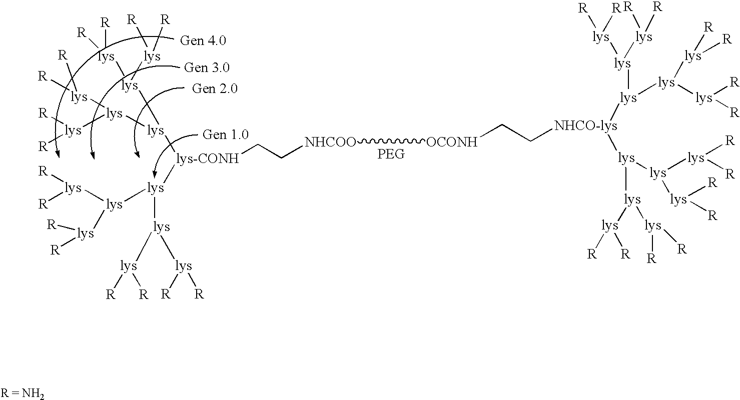

N,N′-Dicyclohexyl carbodiimide (0.49 g, 2.4 mmol) was dissolved in 5 ml of CH₂Cl₂. With stirring, to this cold solution was dropwise added a cooled solution of N², N⁶-di-t-Boc-L-Lys (0.83 g, 2.4 mmol) in CH₂Cl₂ (10 mL). The reaction temperature was kept below −10° C. Several minutes later, the reaction mixture turned turbid, then was continued to stir for 5 minutes. Immediately afterwards, a cooled solution of PEG16000 bisamine (2.07 g, 0.608 mmol NH₂) and DIPEA (0.39 g, 3 mmol) in 10 ml of CH₂Cl₂ ,was added with stirring at −5° C. This coupling reaction was continued at −5° C. for 0.5 h and room temperature for 20 h. White precipitate was filtered and washed by CH₂Cl₂, the filtrate was evaporated under reduced pressure to give a thick syrup. After the dissolution of the syrup in 3 ml of CH₂Cl₂, 120 ml of anhydrous ether was added. Standing half an hour during which scratching was made occasionally, a white precipitate separated and gradually solidified. This procedure was repeated twice so as to remove DIPEA and excess di-t-Boc-lysine from the crude product. The compound was washed profusely by ether and dried in vacuum. A white, loose solid (PEG6000-carbamate-Gen0.5) was afforded in a yield of 87% (1.93 g). Ninhydrin test: negative.

PEG6000-carbamate-(Gen0.5 (1.8 g, 0.247 mmol) was dissolved in 15 ml of CH₂Cl₂ and cooled to 0-5° C. With stirring, 15 ml of TFA was added slowly while the reaction temperature was kept below 20° C. After the TFA addition, the reaction was continued at room temperature for 2-3 h. After the reaction went to completion, the mixture was evaporated under reduced pressure below 40° C., giving a slightly yellow syrup. Anhydrous ether was added. Standing 1-2 h at room temperature, the syrup solidified. The dissolution/precipitation procedure was repeated twice. The white precipitate was collected, washed with ether and dried in vacuum to give 1.78 g of a white powder as PEG6000-carbamate-Gen1.0 (TFA salt, yield 98%).

The following procedures from Gen1.5 through Gen 4.0 are similar. In the coupling steps, the mole ratio of t-Boc-lysine to amino group was kept at 4-6. Yields of coupling steps ranged from 85 to 92%, while those of the deprotection steps varied from 90 to 98%.

PEG6000-carbamate-Gen4.0 crude product was purified by size-exclusion chromatography (Sephadex G-50).

Overall yield 43% (8 steps). Elemental analysis (%):

| | C | H | N |
|---|---|---|---|
| Found | 45.80 | 7.21 | 6.52 |
| Theoretical | 46.18 | 7.09 | 6.34 |

e) Hydroxyl Protection of Iobitridol by Acetalization (TIP-IOB)

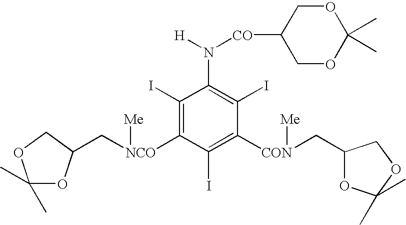

Iobitridol powder (16.70 g, 20 mmol) was added in 50 ml of DMF. With stirring 12.5 g of 2,2′-dimethoxylpropane(120 mmol, DMP) and 100 mg of p-toluenesulfonic acid monohydrate were then added, consecutively. The resulting solution was brought up to 90° C. and stirred at this temperature for 0.5 h in which the acetalization reaction was shown complete by TLC. DMF and excess DMP were removed by vacuum distillation. A crude product was obtained as a yellow syrup. Further purification was followed by silica gel chromatography using CHCl₃ then CHCl₃/CH₃OH 8:1 as the eluting solvent, 18.7 g of slightly yellow solid, namely the tri-isopropylidene derivative of Iobitridol (TIP-IOB), was obtained.

Yield 98%. Elemental analysis (%):

| | C | H | N | I |
|---|---|---|---|---|
| Found | 36.30 | 4.05 | 4.68 | 39.57 |
| Theoretical | 36.46 | 4.22 | 4.40 | 39.85 |

f) N-Alkylation With Ethyl Bromoacetate (TIP-IOB-CH₂COOEt)

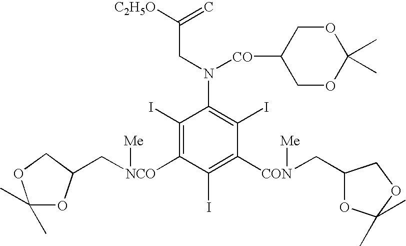

The compound (10.00 g, 10.47 mmol) prepared in 1e) was dissolved in 15 ml of anhydrous tetrahydrofuran (distilled from Na). 7.70 ml of 1.90 Ni sodium methoxylate (prepared from Na and methanol, titrated by potassium hydrogen phthalate) was added with stirring. Upon addition, the clear yellow solution turns orange. After 5 min, 3.8 g of ethyl bromoacetate (20.94 mmol) was added. White precipitate appeared and the mixture was stirred for 3 h at room temperature. After filtration, the tetrahydrofuran solution was washed to neutral by water and dried over anhydrous MgSO₄. The solution was evaporated to give a yellow oil as crude product. Following purification was made by silica get chromatography, eluting first with CHCl₃ to remove excess ethyl bromoacetate, then eluting with CHCl₃/CH₃OH (10:1, v/v) to get the pure title compound as a slightly yellow solid (10.30 g)

Yield 95%. Elemental analysis (%):

| | C | H | N | I |
|---|---|---|---|---|
| Found | 38.25 | 4.72 | 4.18 | 36.20 |
| Theoretical | 38.06 | 4.45 | 4.03 | 36.56 |

g) Hydrolysis of Ethyl Ester (TIP-IOB-CH₂COOH)

Methanol (18 ml) was added to dissolve 10.5 3(10.09 mmol) of the ester obtained in 1f). With stirring, 18 ml of 2 N NaOH solution was added. The reaction mixture was stirred for 2.5 h at room temperature. Methanol was removed by evaporation at a 35° C. water bath. The residue was dissolved in 80 ml of water and adjusted to pH 3.5 by 2 N HCl, a lot of white precipitate appeared. Saturated NaCl solution (80 ml) was added, then 3×100 ml of chloroform was used to extract the carboxylic acid. The extracts were dried over magnesium sulfate overnight and evaporated to give 9.37 g of yellowish solid.

Yield 91.7%. Elemental analysis (%):

| | C | H | N | I |
|---|---|---|---|---|
| Found | 36.44 | 4.30 | 4.25 | 37.84 |
| Theoretical | 36.74 | 4.18 | 4.15 | 37.57 |

h) Preparation of N-Hydroxysuccinimide Ester of Triiodinated Carboxylic Acid (TIP-IOB-CH₂CO—OSu)

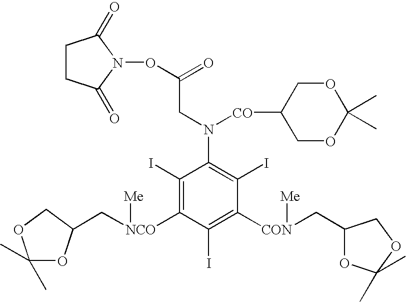

The carboxylic acid (9.12 g, 9.0 mmol) prepared in 1 g) and N-hydroxysuccinimide (1.04 g, 9.0 mmol) were dissolved in 40 ml of chloroform. Cooled to −5-0° C. dicyclohexylcarbodiimide (1.85 g, 9.0 mmol) in 5 ml of chloroform was added. The reaction continued for half an hour at 0° C. and r.t. for 24 h. White precipitate was filtered and the filtrate was evaporated to give 9.89 g of active ester.

Yield 99%. Elemental analysis (%):

| | C | H | N | I |
|---|---|---|---|---|
| Found | 37.66 | 4.17 | 4.84 | 34.52 |
| Theoretical | 37.86 | 4.08 | 5.05 | 34.28 |

i) PEG6000-carbamate-Gen4-IOB

(Structure similar to that in FIG. 4 of U.S. Provisional Patent Application 60/785,260 filed Mar. 23, 2006, but with PEG6000 instead of PEG12000)

PEG6000-carbamate-Gen4.0 amplifier (TFA salt, 0.76 g, 172 mmol NH₂ groups) and 0.66 g of DIPEA (5.13 mmol) were dissolved in 20 ml of DMF, 5.70 g of active ester (5.13 mmol) prepared in 1 h) was added dan the reaction last for 72 h at room temperature. Cooled to room temperature, a small amount of 2-aminoethanol (4 mmol) was added to destroy the excess un-reacted active ester. Solvents were removed by vacuum distillation, the residue obtained was dissolved in 150 ml of chloroform and washed by saturated saline (2×20 ml) to remove water-soluble byproduct N-hydroxysuccinimide. The chloroform solution was dried, and evaporated. The syrup thus obtained was dissolved in 3 ml of methanol and precipitated by 100 ml of anhydrous ether/petroleum ether (3:1). A yellowish syrup was obtained. The hydroxy-protected conjugate obtained above was treated by 40 ml of 60%(v/v) aqueous TFA at room temperature for 3 h, a clear and lightly yellow solution was obtained containing the deprotected macromolecular conjugate. After the pH was adjusted to 7, it was dialyzed in a dialysis tubing (cutoff MW 3500) against 4-litre of distilled water for 2 h. The dialyzate was concentrated, then underwent preparative SEC on a Sephadex G-50 column with 0.1 M ammonium acetate as the eluting solvent. Collected macromolecular fractions were lyophilized to give 1.84 g of white and loose material PEG6000-carbamate-Gen4.0-IOB.

Yield 90%. Elemental analysis (%):

| | C | H | N | I |
|---|---|---|---|---|
| Found | 36.64 | 4.98 | 5.54 | 31.20 |
| Theoretical | 36.93 | 4.86 | 5.82 | 31.66 |

### Example 2

Preparation of Peg12000-Disulfide-Gen4-(β-Ala-lob) Conjugate

a) Bis(4-Nitrophenyl Carbonate)-PEG12000 Preparation

Dried PEG12000 (Mn 12160, 7.54 g, 0.62 mmol) was dissolved in 40 ml of anhydrous CH2Cl2 and 15 ml of dry pyridine then cooled to 0° C. To it was added 1.0 g of 4-nitrophenyl chloroformate (4.96 mmol) and 80 mg of DMAP. At 0° C. this mixture was stirred for 8 h. The reaction mixture was evaporated and precipitated by 80 ml of ether. Standing 0.5 h, the syrupy precipitate solidified, which was filtered and washed by ether. The crude product was dissolved in 1-2 ml of CH2Cl2 and precipitating it by 60 ml of anhydrous ether, this procedure was repeated twice. A white powder (7.58 g) was finally obtained.

Yield 98%. Elemental analysis (%):

| | C | H | N |
|---|---|---|---|
| Found | 54.14 | 9.12 | 0.28 |
| Theoretical | 54.42 | 8.97 | 0.22 |

b) α, ω-Bis(N-t-Boc-ethyldithioethylcarbamoyl)-PEG12000

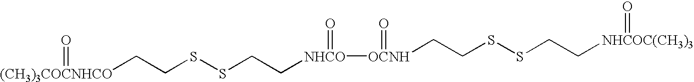

Under argon atmosphere and in an ice-water bath, a solution of di-t-butyl carbonate (4.64 g, 26.7 mmol) in 20 ml of methanol was added dropwise to 80 ml of methanol solution of cystamine hydrochloride (24 g, 106.6 mmol) in the presence of NaOH (8.6 g, 215 mmol) with stirring. After the addition, the reaction was continued for half an hour at 0° C. and for 20 h at room temperature. The solvent was removed by evaporation, the residue was mixed with 300 ml of water and extracted by chloroform (150 ml×3). The chloroform solution was back-washed by water (100 ml×2) and dried over anhydrous magnesium sulfate, and evaporated to dryness. The crude product was purified by silica get chromatography, eluting firstly with chloroform then chloroform/methanol (10:1), followed by di-N-t-butyloxycarbonyl (Boc) cystamine byproduct, the mono-N-t-Boc cystamine was eluted out (yield 5.0 g, 74%).

To a solution of mono-N-t-Boc-cystamine (0.78 g, 3.08 mmol) in 15 ml of CHCl₃ and 0.6 g of DIPEA (4.64 mmol) was added dropwise 25 ml of chloroform solution containing 0.38 mmol of the compound prepared in 2a) at 0° C. This reaction was allowed to continue 24 h at room temperature. The resulting mixture was evaporated and precipitated by 100 ml of ether, further purified by several dissolution-precipitation cycles using CHCl₃ and ether. A white powder (4.59 g) was obtained.

Yield 97%. Elemental analysis (%):

| | C | H | N | S |
|---|---|---|---|---|
| Found | 53.64 | 8.75 | 0.56 | 1.10 |
| Theoretical | 53.97 | 9.01 | 0.44 | 1.00 |

c) PEG12000-Disulfide-GEN0.0

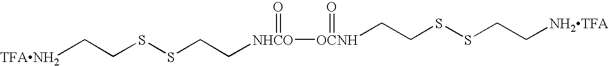

t-Boc-protected biscarbamate (5.55 g, 0.446 mmol) obtained above was dissolved in 15 ml of CH₂Cl₂ and cooled to 0° C., to which was added 15 ml of TFA. This mixture was stirred at 0° C. for 30 min and room temperature for another 2 h. Evaporation of the solution gave an oil which was solidified while standing at r.t. for several hours after adding 80 ml of anhydrous ether. The crude product was treated twice by dissolution-precipitation with CH₂Cl₂/ether. A white powder product (5.38 g) was obtained.

Yield 97%. Elemental analysis (%):

| | C | H | N | S |
|---|---|---|---|---|
| Found | 53.05 | 9.24 | 0.52 | 1.13 |
| Theoretical | 53.34 | 8.92 | 0.44 | 1.01 |

d) PEG12000-Disulfide-Gen4.0

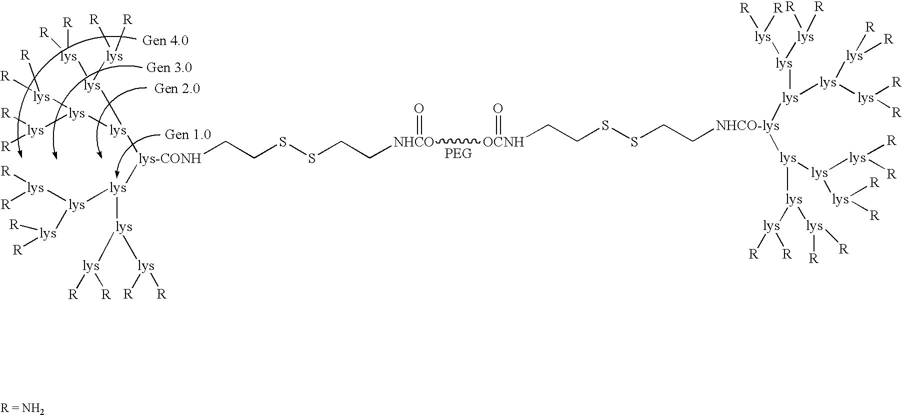

N,N′-Dicyclohexyl carbodiimide (0.49 g, 2.4 mmol) was dissolved in 5 ml of CH₂Cl₂. With stirring, to this cold solution was dropwise added a cooled solution of N², N⁶-di-t-Boc-L-Lysine (0.83 g, 2.4 mmol) in CH₂Cl₂ (10 mL). The reaction temperature was kept below −10° C. Several minutes later, the reaction mixture turned turbid, then was continued to stir for 5 minutes. Immediately afterwards, a cooled solution of PEG12000 bisamine (3.82 g, 0.304 mmol, 0.608 mmol NH₂) and DIPEA (0.39 g, 3 mmol) in 10 ml of CH₂Cl₂ was added with stirring at −5° C. This coupling reaction was continued at −5° C. for 0.5 h and room temperature for 20 h. White precipitate was filtered and washed by CH₂Cl₂, the filtrate was evaporated under reduced pressure to give a thick syrup. After the dissolution of the syrup in 3 ml of CH₂Cl₂, 160 ml of anhydrous ether was added. Standing half an hour during which scratching was made occasionally, a white precipitate separated and gradually solidified. This procedure was repeated twice so as to remove DIPEA and excess di-t-Boc-lysine from the crude product. The compound was washed profusely by ether and dried in vacuum. A white, loose solid (PEG12000-disulfide-Gen0.5) was afforded in a yield of 89% (3.52 g). Ninhydrin test: negative.

PEG12000-disulfide-Gen0.5 (3.21 g, 0.247 mmol) was dissolved in 25 ml of CH₂Cl₂ and cooled to 0-5° C. With stirring, 15 ml of TFA was added slowly while the reaction temperature was kept below 20° C. After the TFA addition, the reaction was continued at room temperature for 2-3 h. After the reaction went to completion, the mixture was evaporated under reduced pressure below 40° C., giving a slightly yellow syrup. Anhydrous ether was added. Standing 1-2 h at room temperature, the syrup solidified. The dissolution/precipitation procedure was repeated twice, the precipitate was collected, washed with ether and dried in vacuum to give 3.03 g of a white powder as PEG12000-disulfide-Gen1.0 (TFA salt, yield 94%).

The following procedures from Gen1.5 through Gen 4.0 are similar. In the coupling steps, the mole ratio of t-Boc-lysine to amino group was kept at 4-6. Yields of coupling steps ranged from 86 to 94%, while those of the deprotection steps varied from 92 to 98%.

PEG12000-disulfide-Gen4.0 crude product (TFA salt) was purified by size-exclusion chromatography (Sephadex G-50).

Overall yield 48% (8 steps). Elemental analysis (%):

| | C | H | N |
|---|---|---|---|
| Found | 47.96 | 7.92 | 4.64 |
| Theoretical | 48.37 | 7.66 | 4.48 |

e) β-Alanine Derivative of TIP-IOB-CH₂CO—OSu

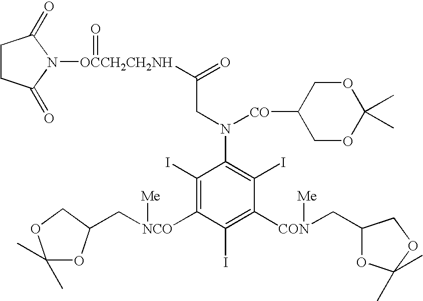

TIP-IOB-CH₁CO—OSu, the compound prepared under 1 h) (20 mmol, 22.2 g), was dissolved in 80 ml of chloroform followed by addition of 40 ml of chloroform solution of β-alanine methyl ester hydrochloride (2.06 g, 20 mmol) and 22 mmol of DIPEA. The reaction was continued for 2 h. The resulting mixture was washed by water (40 ml×3), dried over anhydrous magnesium sulfate, evaporated to give a slightly yellow solid. The methyl ester thus obtained was dissolved in the mixture of methanol (40 ml) and 2 N NaOH (40 ml). The reaction mixture was stirred for 2.5 h at room temperature. Methanol was removed by evaporation at a 35° C. water bath. The residue was dissolved in 80 ml of water and adjusted to pH 3.5 by 2 N HCl, a lot of white precipitate appeared. Saturated NaCl solution (80 ml) was added, then 3×160 ml of chloroform was used to extract the carboxylic acid. The extracts were dried over magnesium sulfate overnight and evaporated to give a yellowish solid. This carboxylic acid (18 mmol) and N-hydroxysuccinimide (18 mmol) were dissolved in 150 ml of chloroform, DCC (18 mmol) in 20 ml of chloroform was added. The reaction lasted for 24 h at room temperature. After the reaction, the mixture was filtered and evaporated to dryness, yielding) a yellowish solid as an active ester (21.4 g).

Yield 89%. Elemental analysis (%):

| | C | H | N | I |
|---|---|---|---|---|
| Found | 38.50 | 4.41 | 5.76 | 32.51 |
| Theoretical | 38.63 | 4.27 | 5.93 | 32.22 |

f) PEG12000-Disulfide-Gen4-β-Ala-IOB)

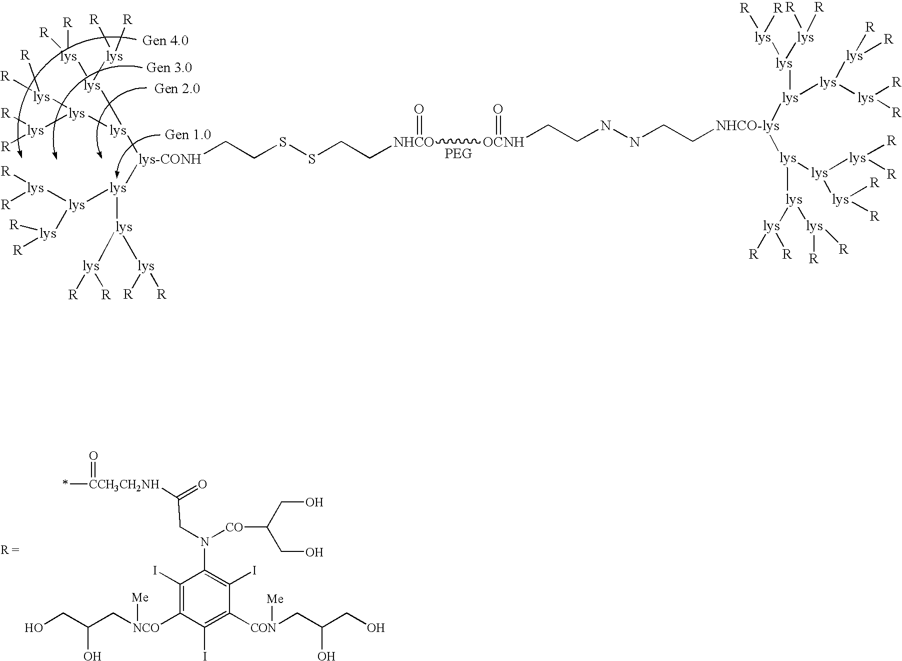

PEG1200-disulfide-Gen4.0 amplifier (TFA salt 1.05 g, 1.72 mmol NH₂ groups) and 0.66 of DIPEA (5.13 mmol) were dissolved in 30 ml of DMF. The active ester (6.06 g, 5.13 mmol) prepared in 2e) was added. The reaction lasted for 72 h at room temperature. Cooled to room temperature, a small amount of 2-aminoethanol (4 mmol) was added to destroy the excess un-reacted active ester. Solvents were removed by vacuum distillation, the residue obtained was dissolved in 200 ml of chloroform and washed by saturated saline (2×30 ml) to remove water-soluble byproduct N-hydroxysuccinimide. The chloroform solution was dried, and evaporated. The syrup thus obtained was dissolved in 3 ml of methanol and precipitated by 100 ml of anhydrous ether/petroleum ether (3:1). A yellowish syrup was obtained. The hydroxy-protected conjugate obtained above was treated by 40 ml of 60% (v/v) aqueous TFA at room temperature for 3 h, a clear and lightly yellow solution was obtained containing the deprotected macromolecular conjugate. After the pH of the solution above was adjusted to 7, it was dialyzed using a dialysis tubing (cutoff MW 3500) for 2 h against 4-litre of distilled water. The dialyzate was concentrated, then underwent preparative SEC on a Sephadex G-50 column with 0.1 M ammonium acetate as the eluting solvent. Collected macromolecular fractions were lyophilized to give 2.19 g of white and loose material PEG12000-disulfide-Gen4.0-(β-Ala-IOB).

Yield 91%. Elemental analysis (%):

| | C | H | N | I |
|---|---|---|---|---|
| Found | 39.30 | 5.64 | 5.94 | 25.85 |
| Theoretical | 39.71 | 5.50 | 5.77 | 26.12 |

### Example 3

Preparation Of Peg12000-Ester-Gen3-lox Conjugate

a) α, ω-Bis(N-t-Boc-β-alanynl)-PEG12000

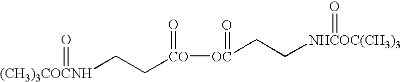

N-t-Boc β-alanine (0.95 g, 5 mmol) in 10 ml chloroform was added slowly to a 15 ml of chloroform solution containing DCC (1.03 g, 5 mmol) at −5° C. Five minutes later, dried PEG12000 (6.0 g, 0.5 mmol, 1.0 mmol hydroxyl groups) and 20 mg of N,N-dimethylaminopyridine were added. This reaction was allowed to continue 24 h at room temperature. The resulting mixture was evaporated and precipitated by 160 ml of anhydrous ether, further purified by several dissolution-precipitation cycles using CHCl₃ and ether. A white powder (5.74 g) was obtained.

Yield 93)%. Elemental analysis (%):

| | C | H | N |
|---|---|---|---|
| Found | 54.82 | 8.85 | 0.27 |
| Theoretical | 54.56 | 9.13 | 0.22 |

b) PEG12000-Ester-Gen0.0

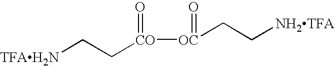

PEG12000 ester (5.55 g, 0.45 mmol) obtained above was dissolved in 15 ml of CH₂Cl₂ and cooled to 0° C. TFA (15 ml) was added with stirring. This reaction lasted for 30 min at 0° C. and then 2 h at room temperature. Evaporation of the solution gave an oil which solidified while standing at r.t. for several hours after adding 80 ml of anhydrous ether. The crude product was purified by dissolution-precipitation with CH₂Cl₂/ether twice. A white powder (5.46 g) was obtained.

Yield 98%. Elemental analysis (%):

| | C | H | N |
|---|---|---|---|
| Found | 53.52 | 9.17 | 0.28 |
| Theoretical | 53.87 | 8.99 | 0.22 |

c) PEG12000-Ester-Gen3.0

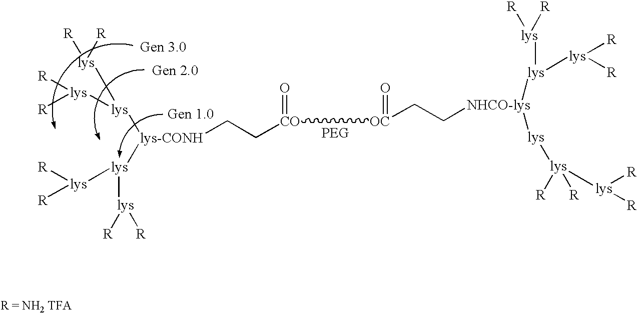

N,N′-Dicyclohexyl carbodiimide (0.49 g, 2.4 mmol) was dissolved in 5 ml of CH₂Cl₂. With stirring, to this cold solution was dropwise added a cooled solution of N², N⁶-di-t-Boc-L-lysine (0.83 g, 2.4 mmol) in CH₂Cl₂ (10 mL). The reaction temperature was kept below −10° C. Several minutes later, the reaction mixture turned turbid, then was continued to stir for 5 minutes. Immediately, a cooled solution of PEG12000-ester-Gen0.0 (3.81 g, 0.304 mmol, 0.608 mmol NH₂) and DIPEA (0.39 g, 3 mmol) in 10 ml of(CH₂Cl₂ was added with stirring at −5° C. This coupling reaction was continued at −5° C. for 0.5 h and room temperature for 20 h. White precipitate was filtered and washed by CH₂Cl₂, the filtrate was evaporated under reduced pressure to give a thick syrup. After the dissolution of the syrup in 3 ml of(CH₂Cl₂, 160 ml of anhydrous ether was added. Standing half an hour during which scratching was made occasionally, a white precipitate separated and gradually solidified. This procedure was repeated twice so as to remove DIPEA and excess di-t-Boc-lysine from the crude product.

The compound was washed profusely by ether and dried in vacuum. A white, loose material (PEG12000-disulfide-Gen0.5) was afforded in a yield of 89% (3.51 g). Ninhydrin test: negative.

PEG12000-ester-Gen0.5 (3.20 g, 0.247 mmol) was dissolved in 25 ml of CH₂Cl₂ and cooled to 0-5° C. With stirring, 15 ml of TFA was added slowly while the reaction temperature was kept below 20° C. After the TFA addition, the reaction was continued at room temperature for 2-3 h. After the reaction went to completion, the mixture was evaporated under reduced pressure below 40° C., giving a slightly yellow syrup. Anhydrous ether was added. Standing 1-2 h at room temperature, the syrup solidified. The dissolution/precipitation procedure was repeated twice. The precipitate was collected, washed with ether and dried in vacuum to give 3.03 g of a white powder as PEG12000-ester-Gen1.0 (TFA salt, yield 94%).

The following procedures from Gen1.5 through Gen 3.0 are similar. In the coupling steps, the mole ratio of t-Boc-lysine to amino group was kept at 4-6. Yields of coupling steps ranged from 85 to 94%, while those of the deprotection steps varied from 90 to 97%.

PEG12000-ester-Gen3.0 crude product (TFA salt) was purified by size-exclusion chromatography (Sephadex G-50).

Overall yield 53% (6 steps). Elemental analysis (%):

| | C | H | N |
|---|---|---|---|
| Found | 51.40 | 7.96 | 2.92 |
| Theoretical | 50.84 | 8.23 | 2.64 |

d) di-isopropylidene Ioxilan (DIP-IOX)

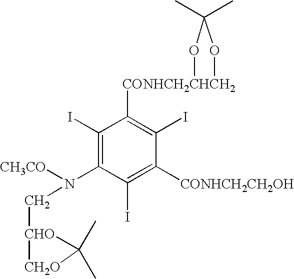

To a stirred solution of Ioxilan (dry powder, 15.80 g, 20 mmol) in anhydrous DMF (50 ml), 2,2′-dimethoxylpropane (DMP 10.42 g, 100 mmol) and p-toluenesulfonic acid monohydrate (TsOH—H₂O, 100 mg) were added. The resulting solution was brought up to 90° C. and stirred at this temperature for 0.5 h. Completion of the reaction was confirmed by TLC. Cooled to room temperature, the reaction mixture was stirred overnight with 2.0 g of dry AG 1 X10 anionic exchange resin (OH⁻ type). The solvent DMF, excess reactant DMP, and byproduct methanol in the reaction were removed by vacuum distillation. A crude product was obtained as a slightly yellow syrup. Further purification was performed by silica gel chromatography using CHCl₃/CH₃OH 20:1 then CHCl₃/CH₃OH 10:1 as the eluting solvent. A slightly yellow solid, di-isopropylidenyl Ioxilan (DIP-IOX) was afforded (16.88 g).

Yield 97% Elemental analysis (%):

| | C | H | N | I |
|---|---|---|---|---|
| Found | 33.38 | 3.97 | 5.16 | 43.35 |
| Theoretical | 33.09 | 3.70 | 4.82 | 43.70 |

e) Activation of di-isopropylidene Ioxilan

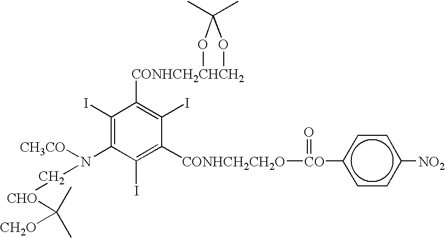

Di-isopropylidene Ioxilan (10 g, 11.5 mmol) was dissolved in 30 mL of anhydrous dichloromethane. Following the addition of 5 ml of pyridine, a solution of 4-nitrophenylchloroformate (2.32 g, 11.5 mmol) in 20 mL of anhydrous dichloromethane was added dropwise with stirring at room temperature. A small amount of DMAP 100 mg was added as the catalyst. This reaction was continued for 2 h. After the reaction, the mixture was concentrated in vacuum to remove solvents. The residue was re-dissolved in 12 mL of dichloromethane and precipitated by 400 mL of 1:1 anhydrous ether and petroleum ether. This procedure was repeated once to give 10.93 g of the carbonate product. Ratio of [nitrophenyl]/[Ioxilan]=0.98 (UV analysis following complete hydrolysis).

Yield 92%. Elemental analysis (%):

| | C | H | N | I |
|---|---|---|---|---|
| Found | 35.72 | 3.48 | 5.62 | 36.43 |
| Theoretical | 35.93 | 3.40 | 5.41 | 36.74 |

f) Preparation of PEG12000-ester-Gen3-IOX Conjugate

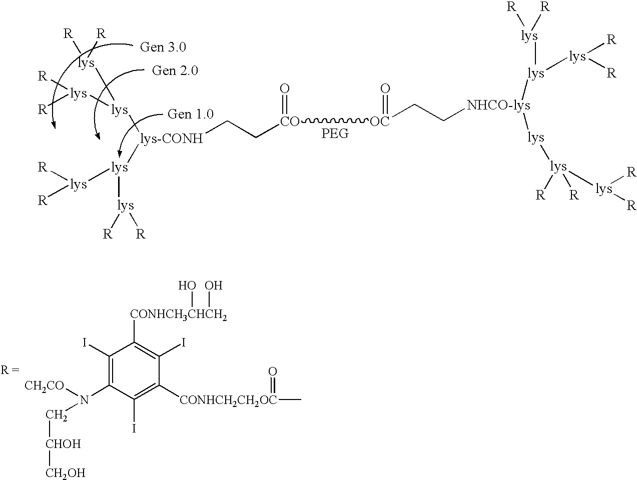

PEG12000-ester-Gen3 TFA salt (3.18 g, 3.2 mmol NH₂) prepared under d) and DIPEA (2.1 g) were dissolved in DMF (60 ml). To this solution was added the active carbonate of DIP-IOX (9.85 g, 9.6 mmol) in three portions with stirring. The reaction was continued for 60 h at room temperature. After the reaction, DMF was removed by vacuum distillation. The residue was dissolved in chloroform (8 ml) and precipitated by anhydrous ether (300 ml). This procedure was repeated once. A yellowish solid thus obtained was suspended in 50% aqueous acetic acid (60 ml), and underwent a deprotection reaction at 80° C. for 36-48 h. After the completion of this reaction, the mixture was neutralized to pH 7 and dialyzed against distilled water (2×2.1) for 2×3 h. The dialyzate was concentrated and underwent SEC purification procedure using Sephadex G-50. A white and loose material was obtained after lyophilization of the collected fractions containing the final product (4.67g).

Yield 86%. Elemental analysis (%):

| | C | H | N | I |
|---|---|---|---|---|
| Found | 41.57 | 6.29 | 4.24 | 22.13 |
| Theoretical | 41.82 | 6.07 | 4.02 | 22.42 |

### Example 4

PEG8000-carbamate-Gen4-IOB (Coupling in Aqueous Phase)

a)PEG8000-carbamate-Gen4

Its synthesis was carried out using the same method described in 1a)-1d), with PEG8000 (Mn 8150) as the starting compound instead of PEG6000. In the coupling steps, the mole ratio of t-Boc-lysine to amino group was kept at 4-6. Yields of coupling steps ranged from 89 to 96%, while those of the deprotection steps varied from 90 to 95%.

Overall yield 37% (11 steps from PEG8000). Elemental analysis (%):

| | C | H | N |
|---|---|---|---|
| Found | 46.82 | 7.59 | 5.51 |
| Theoretical | 47.07 | 7.31 | 5.67 |

b) TIP-IOB-CH₂CO—NHNH-t-Boc

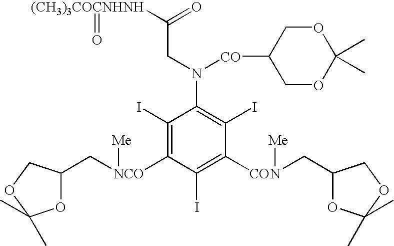

t-Butyl carbazate (0.54 g, 4.0 mmol) was dissolved in chloroform (30 ml), the active ester (4.44 g, 4.0 mmol) prepared under 1 h) were added. The mixture was stirred for 6 h at room temperature. After the reaction, the chloroform solution was diluted by chloroform (70 ml) and washed by water (2×35 ml), dried overnight, and evaporated to dryness, yielding a white solid product (4.15 g).

Yield 92%. Elemental analysis (%):

| | C | H | N | I |
|---|---|---|---|---|
| Found | 38.62 | 4.80 | 5.94 | 33.95 |
| Theoretical | 38.35 | 4.65 | 6.21 | 33.77 |

c) IOB-CH₂CO—NHNH₂

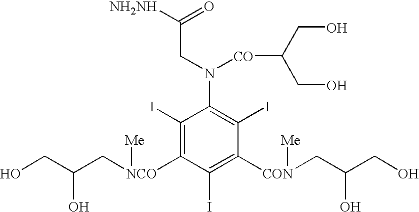

The product (4.0 g, 3.55 mmol) in b) step was dissolved in methanol (25 ml) and mixed with TFA (25 ml). The mixture was stirred for 6 h and evaporated under reduced pressure. The residue was re-dissolved in a small amount of water (2 ml) and lyophilized, giving a white solid (TFA salt, 3.59 g).

Yield 99% (TFA salt), Elemental analysis (%):

| | C | H | N | I |
|---|---|---|---|---|
| Found | 27.96 | 3.48 | 7.13 | 37.55 |
| Theoretical | 28.23 | 3.26 | 6.86 | 37.28 |

d) IOB-CH₂CO—N₃

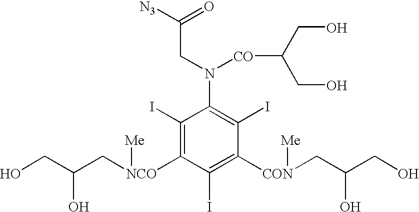

The hydrazide (3.10 g, 3.04 mmol) prepared in c) step was dissolved in 1 N HCl (15 ml), cooled to −5° C. With stirring, a solution of sodium nitrite (1 N, 15 ml) was added dropwise over 10 min. The mixture was stirred for further 30 min at this temperature. The pH was brought to 5 by saturated sodium carbonate at 0° C. This resulting solution should be immediately used in subsequent reaction at low temperature. Its purity was confirmed by reverse phase HPLC.

e) PEG8000-carbamate-Gen4-IOB

A solution of freshly prepared azide above (10 mmol) was added in three portions to a solution of the prepared amplifier under a) (0.99 g, 2 mmol NH₂ groups) in 20 ml of 0.25 M Hepes buffer (pH 8.5) at 0° C. the reaction was continued for 6 h at 0° C. and 48-72 h at room temperature. During this course the pH was maintained between 7.8-8.5 by addition of 0.5 N NaOH when necessary. After the coupling reaction, the solution was neutralized and dialyzed against distilled water (2×2 l) for 2×3 h. The dialyzate was further purified by size exclusion chromatography using Sephadex G-50 gel. A white loose material was obtained after lyophilization (1.98 g). (See structure in FIG. 4 of U.S. Provisional Patent Application 60/785,260 filed Mar. 23, 2006 but with PEG8000 instead of PEG12000)

Yield 79%. Elemental analysis (%):

| | C | H | N | I |
|---|---|---|---|---|
| Found | 37.31 | 5.18 | 5.76 | 29.98 |
| Theoretical | 37.66 | 5.05 | 5.58 | 30.33 |

### Example 5

PEG3400-amide-Gen5-(DTPA-Gd)

a) PEG3400-amide-Gen5 (see FIG. 2 of U.S. Provisional Patent Application 60/785,260 filed Mar. 23, 2006.)

This compound was synthesized in a similar method described in 1d) but with PEG3400 bisamine as the starting material instead, and with one more generation (5^{th} generation) created. In the coupling steps, the mole ratio of t-Boc-lysine to amino group was kept at 4-6. Yields of coupling steps ranged from 82 to 89%, while those of the deprotection steps varied from 88 to 95%.

Overall yield 39% (10 steps from PEG bisamine). Elemental analysis (%):

| | C | H | N |
|---|---|---|---|
| Found | 42.39 | 5.90 | 9.87 |
| Theoretical | 42.07 | 6.04 | 9.53 |

(b) PEG3400-amide-Gen-5-(DTPA-Gd)

Diethylenetriaminepentaacetic acid, namely DTPA (3.93 g, 10 mmol), was dissolved in DMF (30 ml) in the presence of DIPEA (9.7 g). N-hydroxysuccinimide (0.57 g, 5 mmol) in DMF (3 ml), and DCC (1.0 g, 5 mmol) in DMF (7 ml) were added respectively. The reaction was continued for 24 h at room temperature.

This resulting solution was added to a 10 ml DMF solution of PEFG3400-amide-Gen5 (0.72 g, 2.5 mmol NH₂) in the presence of DIPEA (0.65 g). The coupling reaction lasted for 72 h. Solvents were removed by evaporation, a syrupy residue was obtained and re-dissolved in water (30 ml). A 130 ml solution of (d complex of nitriloacetic acid (13 mmol), namely Gd(NTA)₂ was added, the pH was adjusted to 5-6 by 2 N NaOH. Stirring was continued for 6 h at room temperature. The reaction mixture was dialyzed against distilled water (2×3 l) for 2×3 h. After concentration of the dialyzate, the crude product was purified by size exclusion chromatography (Saphadex-50), lyophilized to give 0.72 g of white and loose material (negative in arsenazo III test), with 25 Gd-DTPA moieties covalently attached.

Yield 74%. Elemental analysis (%):

| | C | H | N | Gd |
|---|---|---|---|---|
| Found | 41.63 | 6.05 | 11.06 | 15.32 |
| Theoretical | 42.00 | 6.20 | 11.31 | 15.79 |

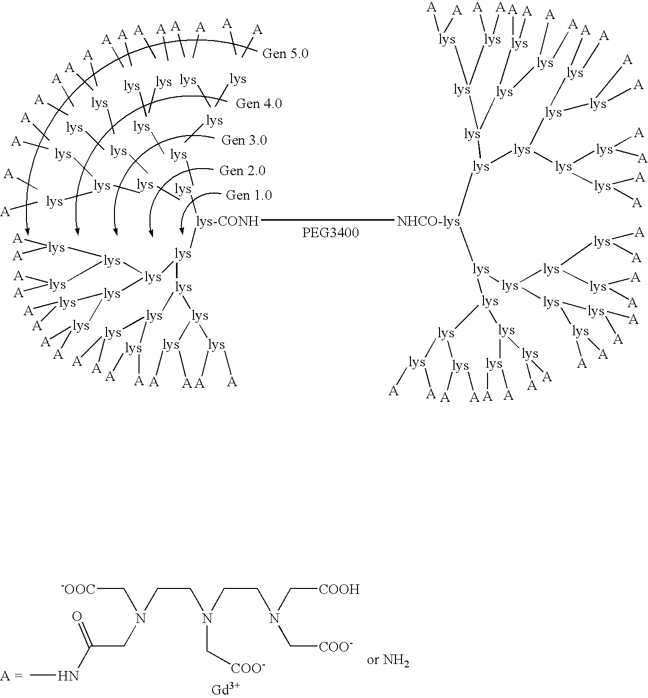

### Example 6

PEG6000-carbamate-Gen4-(Gly-DO3A-Gd)

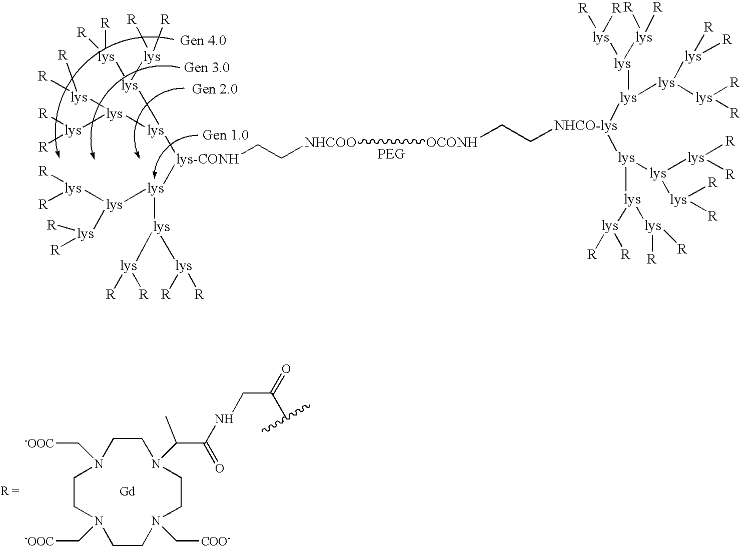

a) Benzyl 2-Bromopropionyl Glycinate

A solution of bromopropionyl chloride (8.58 g, 50 mmol) in chloroform (40 ml)was added dropwise to the solution of benzyl glycinate (8.25 g, 50 mmol) and pyridine (4.5 g) in chloroform (80 ml) at 0° C. After the addition is complete, the mixture was stirred for 3 h at room temperature. The precipitate was filtered off, the solvents were evaporated from the filtrate. A residue thus obtained was dissolved in ether (300 ml) and washed by citrate buffer (pH 3.5, 0.5 M, 2×50 ml), then by water (2×60 ml), dried overnight with anhydrous magnesium sulfate. Evaporation of the ether solution gave the product (12.9 g).

Yield 86%. Elemental analysis (%):

| | C | H | N | Br |
|---|---|---|---|---|
| Found | 48.29 | 4.55 | 4.78 | 26.94 |
| Theoretical | 48.02 | 4.70 | 4.67 | 26.62 |

b) 10-(Benzyl 2-Propionyl Glycinate)-1,4,7-DO3A-tri-t-butyl Ester

To a solution of DO3A-tri-t-butyl ester (5.14 g, 10 mmol) and DIPEA (1.55 g, 12 mmol) in chloroform (30 ml), 20 ml chloroform solution containing benzyl 2-bromopropionylglycinate (3.0 g, 10 mmol) was added dropwise with stirring. The reaction lasted for 24 h at room temperature. Resulting mixture was filtered and diluted by chloroform (100 ml), then washed by water (3×50 ml). After the solution was dried overnight over anhydrous magnesium sulfate, the solvent was evaporated to give a crude product. Silica gel chromatography was conducted using 20:1 chloroform/methanol as the eluting solvent, giving a white solid product (6.02 g).

Yield 82%. Elemental analysis (%):

| | C | H | N |
|---|---|---|---|
| Found | 61.88 | 8.43 | 9.62 |
| Theoretical | 62.19 | 8.65 | 9.54 |

c) 10-(Succinimidyl 2-Propionyl Glycinate)-1,4,7-DO3A-tri-t-butyl Ester

The benzyl ester (5 g, 6.81 mmol) was dissolved in methanol (40 ml), 5% Pd/C catalyst (1.0 g) was added under argon atmosphere. Hydrogen was then bubbled into the mixture with stirring. The hydrogenation lasted for 8 h. After the reaction went to completion, the catalyst was filtered off, methanol was evaporated to dryness. The acid thus obtained was re-dissolved in chloroform (40 ml), N-hydroxysuccinimide (0.78 g, 6.80 mmol) and DCC (1.4 g, 6.80 mmol). The reaction was continued for 24 h at room temperature. The urea precipitate was filtered, the filtrate was evaporated to give a white solid as the succinimidyl ester (4.74 g).

Yield 94%. Elemental analysis (%):

| | C | H | N |
|---|---|---|---|
| Found | 56.45 | 7.87 | 11.61 |
| Theoretical | 56.74 | 8.16 | 11.34 |

d) PEG6000-carbamate-Gen4-(DO3A-Gd)

With stirring, the active ester (8.89 g, 12 mmol) prepared under c) was added to the solution of PEG6000-carbamate-Gen4 (1.77 g, 4 mmol NH₂, the preparation under 1d)) in DMF (60 ml) in the presence of DIPEA (1.0 g). The reaction was continued for 72 h at room temperature. DMF was removed by vacuum distillation. The residue was diluted by a small volume of chloroform (10 ml) and precipitated with a large amount of ether (300 ml). A syrup was obtained and treated by 2 N HCl in methanol (60 ml) for 3 h with mild stirring. Solvents were evaporated, a macromolecular ligand was obtained. After it was dissolved in water (50 ml) an aqueous solution (150 ml) of Gd complex of nitriloacetic acid (15 mmol), namely Gd(NTA)₂ was added, the pH was adjusted to 5-6 by 2 N NaOH. Stirring was continued for 6 h at room temperature. The reaction mixture was dialyzed against distilled water (2×4 l) for 2×3 h. After concentration of the dialyzate, the crude product was purified further by size exclusion chromatography, giving 3.56 g of white and loose material (negative in arsenazo III test).

Yield 93%. Elemental analysis (%):

| | C | H | N | Gd |
|---|---|---|---|---|
| Found | 42.16 | 6.50 | 10.58 | 16.02 |
| Theoretical | 42.63 | 6.33 | 10.24 | 16.43 |

What is claimed is:

1. A compound having a structure according to Formula (I):
(R³-z-S²-w)-R²-(y-S¹-x)-R¹-(x-S¹-y)-R²-(w-S²-z-R³) (I)
wherein
R¹ is a water-soluble polymeric moiety selected from polyalkylene glycol and derivatives thereof;
R² is a cascade polymer amplifier component having the formula:
U—(NH-Z)ₙ
wherein
U is a reproduction unit of the starting generation, and
Z is a repeating unit of a subsequent generation characterized by a number of generations, wherein said number of generations is selected from 0 to 10; and
n is an integer selected from 2 to 6;
R³ is a signal enhancing group, which is a member selected from a paramagnetic chelate and an iodinated contrast agent;
S¹ and S² are spacer groups independently selected from substituted or unsubstituted straight chain or branched chain C₁-C₁₂ alkyl and substituted or unsubstituted C₃-C₁₂ cycloalkyl, wherein each spacer group optionally comprises one or more oxygen atom, one or more carbonyl group or one or more imino group wherein said imino group is optionally substituted with a carboxymethyl group, and said C₁-C₁₂ alkyl group is optionally mono- or polysubstituted with one or more hydroxyl group, one or more carboxyl group, one or more sulfono group, one or more phosphono group or one or more C₁-C₄ alkoxy group;
X, Y and Z are linker moieties independently selected from amide, carbamate, hydrazide, ureido, thioureido, azo, azido, ester, thioester, carbonate, phosphoester and disulfide.

2. The compound according to claim 1, wherein said polyalkylene glycol is polyethylene glycol.

3. The compound according to claim 2, wherein R¹ has an average molecular weight of about 100 to about 10,000,000 daltons.

4. The compound according to claim 2, wherein R¹ is linear polyethylene glycol with an average molecular weight of about 100 to about 45,000 daltons.

5. The compound according to claim 4, wherein R¹ has an average molecular weight of about 1000 to about 20,000 daltons.

6. The compound according to claim 1, wherein said compound comprises between about 2 to about 2048 of said signal enhancing groups R³.
